(19) United States
(12) Patent Application Publication
(10) Pub. No.: US 2009/0288964 A1
(43) Pub. Date: Nov. 26, 2009

(54) BIOSENSOR WITH CODED INFORMATION AND METHOD FOR MANUFACTURING THE SAME

(76) Inventors: Sung-Kwon Jung, Granger, IN (US); Steven C. Charlton, Osceola, IN (US); Dijia Huang, Granger, IN (US)

Correspondence Address: NIXON PEABODY LLP, 300 S. Riverside Plaza, 16th Floor, CHICAGO, IL, 60606-6613, US

(21) Appl. No.: 12/515,946

(22) PCT Filed: Dec. 3, 2007
(86) PCT No.: PCT/US07/24757

§ 371 Date: May 21, 2009

Related U.S. Application Data
(60) Provisional application No. 60/874,649, filed on Dec. 13, 2006.

(51) Int. Cl. C12Q 1/32; G01N 33/487; G01F 1/64

(52) U.S. Cl. 205/792; 204/403.14; 204/403.01

(57) ABSTRACT

An electrochemical test sensor and method for forming the same for determining the concentration of an analyte in a fluid sample includes a base, a reagent layer, a lid, and a meter contact area that has a plurality of contacts. The meter contacts have a first testing contact, a second testing contact, and at least four coding contacts. At least a first electrical connection forms between the first testing contact and a first one of the plurality of coding contacts. At least a second electrical connection forms between the second testing contact and a second one of the plurality of coding contacts. A plurality of electrical connections forms or are severed between the plurality of adjacent coding contacts. At least one of the connections in the meter contact area is terminated or formed to encode calibration information on the test sensor.

### FIELD OF THE INVENTION

The present invention relates generally to electrochemical test sensors. More particularly, the invention relates to electrochemical test sensors adapted to assist in determining a concentration of at least one analyte, where the test sensors are calibrated.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. The results of such tests may be used to determine what, if any, insulin or other medication needs to be administered. In one type of testing system, test sensors are used to test a fluid such as a sample of blood.

A test sensor contains biosensing or reagent material that reacts with blood glucose. In some mechanisms, the testing end of the sensor is adapted to be placed into the fluid being tested, for example, blood that has accumulated on a person's finger after the finger has been lanced. The fluid may be drawn into a capillary channel that extends into the sensor from the testing end to the reagent material by capillary action so that a sufficient amount of fluid to be tested is drawn into the sensor. The fluid then chemically reacts with the reagent material in the sensor resulting in an electrical signal indicative of the glucose level in the fluid being tested. This signal is supplied to the meter via contact areas located near the rear or contact end of the sensor and becomes the measured output. In other mechanisms, the sensor has a reagent area upon which the blood is applied. The resulting chemical reaction produces a color change. When the sensor is inserted into an instrument, the color change can be optically measured and converted into an equivalent glucose concentration value.

Diagnostic systems, such as blood-glucose testing systems, typically calculate the actual glucose value based on a measured output and the known reactivity of the reagent-sensing element (test sensor) used to perform the test. The reactivity or lot-calibration information of the test sensor may be given to the user in several forms including a number or character that is manually entered into the instrument. Another method for calibrating strips contained within a package is to include a calibration chip within the sensor packaging that is inserted into the test instrument. When plugged into the instrument, the calibration chip's memory element is electrically coupled to the instrument's microprocessor board for directly reading the stored calibration information by the instrument.

These methods suffer from the disadvantage of relying on the user to enter the calibration information, which some users may not enter at all or may input incorrectly. In this event, the test sensor may use the wrong calibration information and thus return an erroneous result. Where a calibration chip is contained within the sensor packaging, the calibration chip can be easily lost or misplaced, resulting in an inability to enter the sensor information via the calibration chip.

Some multiple test sensor cartridge systems use an auto-calibration label that is affixed to a sensor cartridge. The auto-calibration label is read automatically when the cartridge is loaded into the meter and requires no additional user intervention. However, such an auto-calibration method requires a cartridge that can be loaded into the meter, that can provide environmental protection for long-term stability of the stored sensors, and that can provide automated access to the sensors. Thus, the additional complexity of such a system and the cost associated with producing an auto-calibration label make it impractical to use in a single test sensor system.

It would be desirable to provide a single test sensor that may be calibrated based upon the reactivity of the reagents within the test sensor that does not require any action by the user of the test sensor to provide the calibration information to instruments or meters in a reliable manner without the complexity, cost, and constraints of an automated cartridge, and without the need for manual entry of calibration information by the user.

### SUMMARY OF THE INVENTION

According to one embodiment, an electrochemical test sensor for determining the concentration of an analyte in a fluid sample comprises a base, a reagent layer, a lid, and a meter contact area. The meter contact area has a plurality of contacts. A high conductivity electrical connection and a low conductivity electrical connection are formed between adjacent contacts. At least one of the electrical connections in the meter contact area is terminated to encode calibration information on the test sensor.

According to another embodiment, an electrochemical test sensor for determining the concentration of an analyte in a fluid sample comprises a base, a reagent layer, a lid, and a meter contact area that has a plurality of contacts. The meter contacts have a first testing contact, a second testing contact, and at least four coding contacts. At least a first electrical connection forms between the first testing contact and a first one of the plurality of coding contacts. At least a second electrical connection forms between the second testing contact and a second one of the plurality of coding contacts. A plurality of electrical connections form between the plurality of adjacent coding contacts. At least one of the connections in the meter contact area is terminated to encode calibration information on the test sensor.

According to another embodiment, an electrochemical test sensor for determining the concentration of an analyte in a fluid sample comprises a base, a reagent layer, a lid, and a meter contact area that has a plurality of contacts. The meter contacts have a first testing contact, a second testing contact, and at least four coding contacts. At least a first electrical connection is added between the first testing contact and a first one of the plurality of coding contacts. At least a second electrical connection is added between two adjacent ones of the plurality coding contacts.

According to a further embodiment, an electrochemical test sensor for determining the concentration of an analyte in a fluid sample comprises a base, a reagent layer, a lid, and a meter contact area that has a plurality of contacts. A reference electrical connection is formed between adjacent ones of the plurality of contacts. At least one additional electrical connection in the meter contact area encodes calibration information on the test sensor.

According to one process, a method of encoding calibration information onto a single test sensor adapted for use in determining a concentration of at least one analyte in a body fluid provides a test sensor that has a plurality of electrical contacts. Each of the plurality of electrical contacts is electrically connected to at least one adjacent contact via at least one connection. The method determines a reactivity level of the test sensor. The method terminates at least one connection electrically connecting two adjacent contacts. A ratio of conductivities is calculated between pairs of adjacent contacts of the plurality of electrical contacts after the termination of the at least one connection. The ratios of conductivity provide information to a test meter regarding the calibration of the test sensor.

According to another process, a method of encoding calibration information onto a single test sensor adapted for use in determining a concentration of at least one analyte in a body fluid provides a test sensor that has a plurality of electrical contacts. The method determines a reactivity level of the test sensor. The method forms at least one connection electrically connecting two adjacent contacts. A ratio of conductivities is calculated between pairs of adjacent contacts of the plurality of electrical contacts after the at least one connection forms. The ratios of conductivity provide information to a test meter regarding the calibration of the test sensor.

The above summary of the present invention is not intended to represent each embodiment, or every aspect, of the present invention. Additional features and benefits of the present invention are apparent from the detailed description and figures set forth below.

### BRIEF DESCRIPTION OF THE DRAWINGS

FIG. 1 is an exploded perspective view of a test sensor according to one embodiment of the present invention.

FIG. 2a is a top view of electrical contacts in according to one embodiment adapted to be used with the test sensor of FIG. 1.

FIG. 2b is a top view of the electrical contacts of FIG. 2a following the calibration of the electrical contacts.

FIG. 3a is a top view of electrical contacts in according to another embodiment adapted to be used with the test sensor of FIG. 1.

FIG. 3b is a top view of the electrical contacts of FIG. 3a following the calibration of the electrical contacts.

FIG. 4a is a top view of electrical contacts in according to another embodiment adapted to be used with the test sensor of FIG. 1.

FIG. 4b is a top view of the electrical contacts of FIG. 4a following the calibration of the electrical contacts.

FIG. 5a is a top view of electrical contacts in according to another embodiment adapted to be used with the test sensor of FIG. 1.

FIG. 5b is a top view of the electrical contacts of FIG. 5a following the calibration of the electrical contacts.

### DESCRIPTION OF ILLUSTRATED EMBODIMENTS

The present invention is directed to an electrochemical test sensor that is adapted to be placed into a meter or an instrument and assist in determining an analyte concentration in a body fluid sample. The body fluid sample may be collected with a lancing device. Examples of the types of analytes that may be collected include glucose, lipid profiles (e.g., cholesterol, triglycerides, LDL and HDL), microalbumin, hemoglobin A_{1C}, fructose, lactate, or bilirubin. It is contemplated that other analyte concentrations may also be determined. The analytes may be in, for example, a whole blood sample, a blood serum sample, a blood plasma sample, other body fluids like ISF (interstitial fluid) and urine, and non-body fluids. As used within this application, the term “concentration” refers to an analyte concentration, analyte level, activity (e.g., enzymes and electrolytes), titers (e.g., antibodies), or any other measure concentration used to measure the desired analyte.

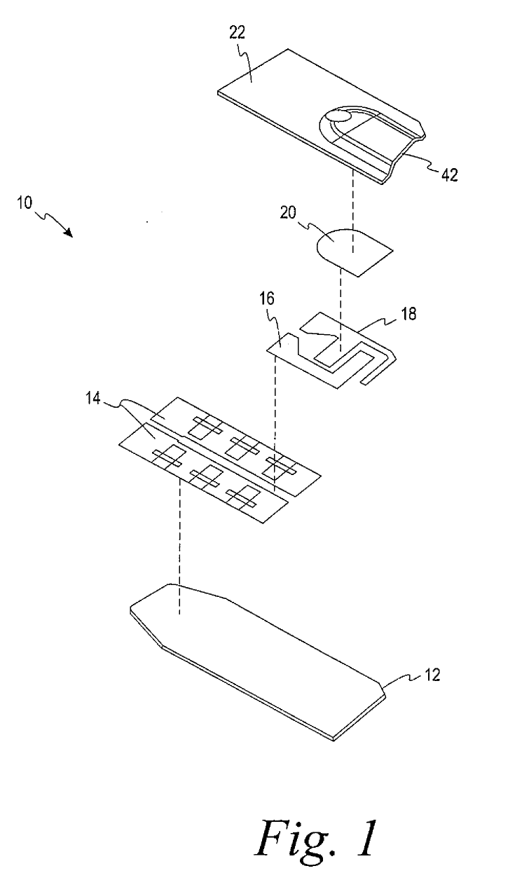

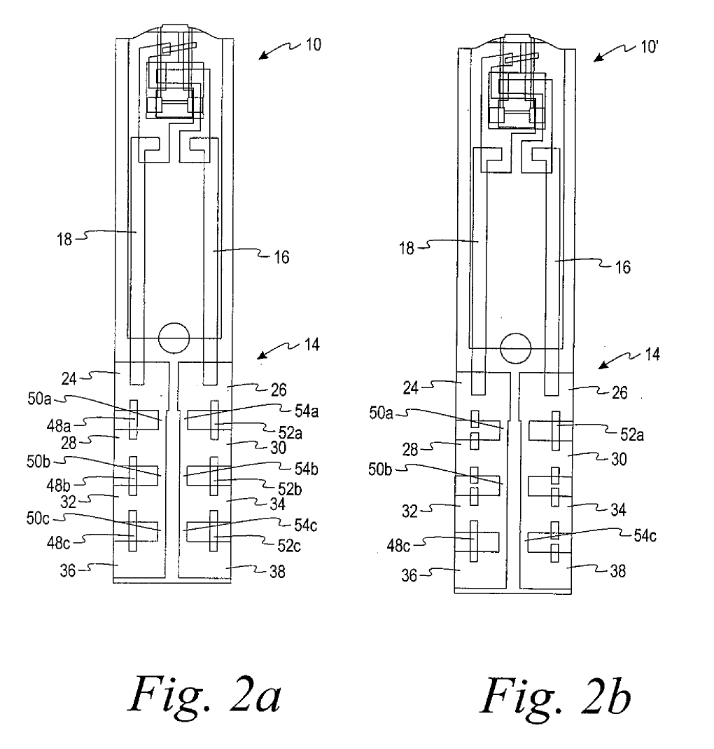

Referring to FIG. 1, a test sensor 10 according to one embodiment is shown. The test sensor 10 includes an insulating base 12, a meter-contact area 14, an electrode pattern (working electrode 16 and counter electrode 18), a reagent layer 20, and a lid 22. The electrochemical test sensor 10 may be printed in sequence such as by screen-printing techniques. It is contemplated that the electrochemical test sensor 10 may be formed by other methods. The meter-contact area 14 of the test sensor 10 has a plurality of electrical contacts 24-38 shown (see FIGS. 2a-2b). The plurality of electrical contacts 24-38 is adapted to interact with similarly arranged contacts on a test meter (not shown) that are adapted to be used in conjunction with the test sensor 10.

The function of the reagent layer 20 is to convert an analyte (e.g., glucose) in the fluid test sample, stoichiometrically into a chemical species that is electrochemically measurable, in terms of electrical current it produces, by the components of the working electrode 16 and counter electrode 18. The reagent layer 20 typically includes an enzyme and an electron acceptor. The enzyme reacts with the analyte to produce mobile electrons on the working and counter electrodes 16, 18. For example, the reagent layer 20 may include glucose oxidase or glucose dehydrogenase if the analyte to be determined is glucose. The enzyme in the reagent layer 20 may be combined with a hydrophilic polymer such as poly(ethylene oxide) or other polymers such as hydroxyethyl cellulose (HEC), carboxymethylcellulose (CMC) and polyvinyl acetate (PVA). The electron acceptor (e.g., ferricyanide salt) carries the mobile electrons to the surface of the working electrode 16.

The working electrode 16 and the counter electrode 18 assist in electrochemically determining the analyte concentration. In one embodiment, the working electrode 16 and the counter electrode 18 comprise a mixture of amorphous and graphite forms of carbon that is chosen to be electrochemically active and provide a low electrical resistance path between the electrodes and the electrical contacts 24-38. In another embodiment, there is an additional layer, or partial layer, that comprises a mixture of carbon and silver beneath the working electrode 16 and the counter electrode 18 to increase the conductivity between the electrodes and the contact area. It is contemplated that the working electrode 16 and the counter electrode 18 may be made of other materials, such as platinum, palladium, or gold that assist in providing an electrical path to the meter or instrument with which they are in operative connection and take part in the electrochemical reaction. The reagent layer 20, as shown in FIG. 1, is directly located on the electrodes 16 and 18. More specifically, there is not an intervening layer (such as a dielectric layer) between the reagent layer and the electrodes 16 and 18. However, it is contemplated that a dielectric layer may be present. Additional electrodes, or subdivisions of existing electrodes, may be present for additional functions, for instance, detection of fill, detection of underfill, and to compensate for sample factors, such as hematocrit or interfering substances.

A three-dimensional lid 22 forms a concave space 42 over the base 12 and the components located thereon eventually form a capillary space or channel. The lid 40 may be formed by embossing a flat sheet of deformable material and then joining the lid 22 to the base 12 in a sealing operation. The material forming the lid 22 may be a deformable polymeric sheet material (e.g. polycarbonate or an embossable grade of polyethylene terphthalate), or a glycol modified polyethylene terephthatalte. It is contemplated that other materials may be used in forming the lid 22. More details on such electrochemical sensors may be found in U.S. Pat. Nos. 5,120,420 and 5,320,732, which are both incorporated herein by reference in their entirety. It is also contemplated that a spacer may be used with a lid to form a channel or capillary space to receive fluid.

Turning now to FIG. 2a, a top view of a portion of the test sensor 10, with the lid 22 removed, is shown as printed during production, depicting the working electrode 16, the counter electrode 18, and the meter-contact area 14. The meter-contact area 14 comprises eight electrical contacts 24-38. The counter electrode 18 is electrically connected to a first contact 24, while the working electrode 16 is electrically connected to a second contact 26. The first contact 24 and the second contact 26 are considered testing contacts, as they are used in determining the analyte level of the sample. A third contact 28, a fourth contact 30, a fifth contact 32, a sixth contact 34, a seventh contact 36, and an eighth contact 38 are considered coding contacts, as they are used in coding a calibration on the test sensor 10. The first contact 24, the third contact 28, the fifth contact 32, and the seventh contact 36 are all connected via a high conductivity connection 48a-48c and a low conductivity connection 50a-50c. Similarly, the second contact 26, the fourth contact 30, the sixth contact 34, and the eighth contact 38 are all connected via a high conductivity connection 52a-52c and a low conductivity connection 54a-54c. It is contemplated that the high conductivity connections 48a-48c and 52a-52c are formed utilizing electrode ink that includes silver. It is contemplated that the low conductivity connections 50a-50c and 54a-54c are formed utilizing electrode ink that contains electrochemically active carbon.

As sensor reactivity may vary from lot to lot, such as variation caused by the reactivity of the reagent, accurate analyte analysis requires calibration of the test sensor, such that the clinical value calculated during the testing of the sample is accurate. Once the reactivity of a lot of sensors has been determined, it may be necessary to place calibration information on the sensors to provide a user with an accurate reading of an analyte level. To place the calibration information on a calibrated test sensor 10′, it is contemplated that various electrical connections within the meter-contact area 14 are severed. As shown in FIG. 2b, the test sensor 10 of FIG. 2a has had various connections severed, which results in the test sensor 10′ that has been coded with calibration information. Conductivity measurements between the contacts are measured and certain ratios of these conductivity measurements are calculated and used to interpret the code and regenerate the calibration information of the calibrated test sensor 10′. Taking ratios of conductivities compensates for variations in conductivity due to normal variations in materials, thicknesses, and processing. For example, the ratio of conductivity between the first contact 24 and the third contact 28 is compared to the conductivity between the second contact 26 and the fourth contact 30. The high conductivity connection 48a of the test sensor 10 has been terminated in the calibrated test sensor 10′. Similarly, the low conductivity connection 54a of the test sensor 10 has been terminated in the calibrated test sensor 10′. Thus, the conductivity between the second contact 26 and the fourth contact 30 is greater than the conductivity between the first contact 24 and the third contact 28. The conductivities measured between the third contact 28 and the fifth contact 32, the fifth contact 32 and the seventh contact 36, the second contact 26 and the fourth contact 30, the fourth contact 30, and the sixth contact 34, the sixth contact 34 and the eighth contact 38 are all divided by (ratioed to) the conductivity measured between the first contact 24 and the third contact 28 to give the ratioed conductivities. The conductivity between the second contact 26 and the fourth contact 30 divided by the conductivity between the first contact 24 and the third contact 28 is termed a reference ratio. The conductivities between the other contacts 28-38 may be coded to calibrate the sensor 10′.

For example, the conductivity between the fourth contact 30 and the sixth contact 34 will be zero, as both the high conductivity connection 52b and the low conductivity connection 54b have been terminated between the two contacts 30, 34. Thus, the ratioed conductivity between the fourth contact 30 and the sixth contact 34 divided by the conductivity between the second contact 26 and the fourth contact 30 will also be zero. A ratioed conductivity of one will be present between the third contact 28 and the fifth contact 32, and the sixth contact 34 and the eighth contact 38. As shown in the calibrated test sensor 10′, the ratioed conductivity between the fifth contact 32 and the seventh contact 36 is generally identical to the reference ratio. This is because only the high conductivity connection 48c remains between the fifth contact 32 and the seventh contact 36.

Thus, three possible ratioed conductivity values are present in the sensor 10 for every pair of contacts: the connection between the contacts may be generally identical to the high conductivity connection, making the ratioed conductivity equivalent to the reference ratio; the connection may be generally identical to the low conductivity connection, making the ratioed conductivity generally one; or the connection between the pair of contacts may be completely terminated, making the ratioed conductivity zero.

A similar calibration may be performed maintaining only the high conductivity connection 48a between the first contact 24 and the third contact 28, and maintaining only the low conductivity connection 54a between the second contact 26 and the fourth contact 30. Therefore, a total of 162 calibrations exist that can be used to calibrate the calibrated sensor 10′.

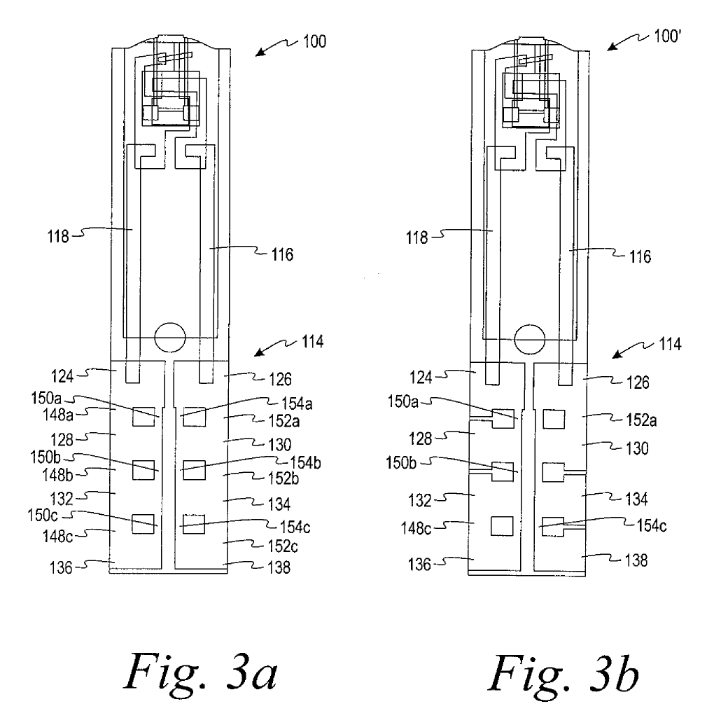

Turning next to FIG. 3a, a test sensor 100 is shown that requires only a single type of ink to obtain a similar calibration as the calibrated sensor 10′. The test sensor 100 has a connection 116 to a working electrode, a connection 118 to a reference or counter electrode, and a meter contact area 114. The meter-contact area 114 comprises eight electrical contacts 124-138. The counter electrode is electrically connected to a first contact 124, while the working electrode is electrically connected to a second contact 126. The first contact 124 and the second contact 126 are considered testing contacts, as they are used in determining the analyte level of the sample. The third contact 128, the fourth contact 130, the fifth contact 132, the sixth contact 134, the seventh contact 136, and the eighth contact 138 are considered coding contacts, as they are used in coding a calibration on the test sensor 100. The first contact 124, the third contact 128, the fifth contact 132, and the seventh contact 136 are all connected via a high conductivity connection 148a-148c and a low conductivity connection 150a-150c. Similarly, the second contact 126, the fourth contact 130, the sixth contact 134, and the eighth contact 138 are all connected via a high conductivity connection 152a-152c and a low conductivity connection 154a-154c. It is contemplated that the high conductivity connections 148a-148c and 152a-152c are significantly wider than the low conductivity connections 150a-150c and 154a-154c. It is additionally contemplated that a single type of electrode ink is utilized for all connections 148a-c and 154a-c.

As shown in FIG. 3b, the test sensor 100 of FIG. 3a has had various connections terminated to result in a calibrated test sensor 100′. As previously described in connection with FIG. 2b, to calibrate the calibrated test sensor 100′, ratios of conductivity are utilized. The ratio of conductivity between the first contact 124 and the third contact 128 is compared to the conductivity between the second contact 126 and the fourth contact 130. The high conductivity connection 148a of the test sensor 100 has been terminated in the calibrated test sensor 100′. Similarly, the low conductivity connection 154a of the test sensor 100 has been terminated in the calibrated test sensor 100′. Thus, the conductivity between the second contact 126 and the fourth contact 130 is greater than the conductivity between the first contact 124 and the third contact 128. The conductivities measured between the other contact pairs 128 and 132, 132 and 136, 126 and 130, 130 and 134, 134 and 138, are all divided by (ratioed to) the conductivity measured between the first contact 124 and the third contact 128 to give the ratioed conductivities. Therefore, the reference ratio is determined by taking the conductivity between the second contact 126 and the fourth contact 130 and dividing by the conductivity between the first contact 124 and the third contact 128. Therefore, the conductivities between the other contacts 128-138 may be coded to calibrate the sensor 100′.

For example, the conductivity between the fourth contact 130 and the sixth contact 134 will be zero, as both the high conductivity connection 152b and the low conductivity connection 154b have been terminated. Thus, the ratioed conductivity between the fourth contact 130 and the sixth contact 134 will also be zero. A ratioed conductivity of one will be present between the third contact 128 and the fifth contact 132, and the sixth contact 134 and the eighth contact 138. As shown in the calibrated test sensor 100′, the ratioed conductivity between the fifth contact 132 and the seventh contact 136 is generally identical to the calibration ratio.

Thus, three possible ratioed conductivity values are present in the sensor 100 for every pair of contacts: the conductivity between the contacts may be generally identical to the high conductivity connection, making the ratioed conductivity generally equal to the calibration ratio; the connection may be generally identical to the low conductivity connection, making the ratioed conductivity generally equivalent to one; or the connection between the pair of contacts may be completely terminated, making the ratioed conductivity zero.

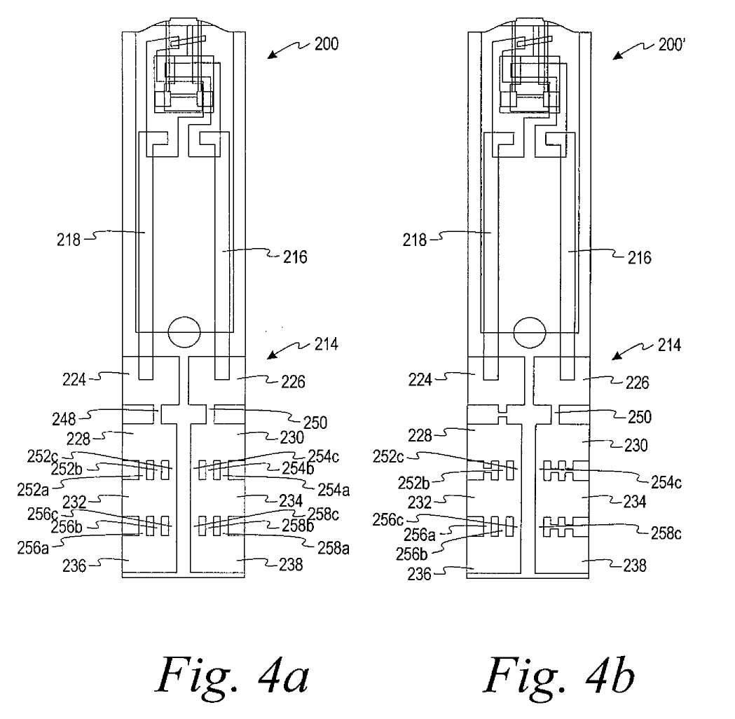

FIG. 4a depicts a test sensor 200 that requires only a single type of ink to obtain a similar calibration as the calibrated sensor 10′. The test sensor 200 has a connection 216 to a working electrode, a connection 218 to a reference or counter electrode, and a meter contact area 214. The meter-contact area 214 comprises eight electrical contacts 224-238. The counter electrode is electrically connected to a first contact 224, while the working electrode is electrically connected to a second contact 226. The first contact 224 and the second contact 226 are considered testing contacts, as they are used in determining the analyte level of the sample. A third contact 228, a fourth contact 230, a fifth contact 232, a sixth contact 234, a seventh contact 236, and an eighth contact 238 are considered coding contacts, as they are used in coding a calibration on the test sensor 200. The first contact 224 and the third contact 228 are connected via a single electrical connection 248. The second contact 226 and the fourth contact 230 are connected via a single electrical connection 250. The third contact 228 and the fifth contact 232 are connected via three electrical connections 252a-252c. Similarly, the fourth contact 230 and the sixth contact 234, the fifth contact 232 and the seventh contact 236, as well as the sixth contact 234 and the eighth contact 238 are all connected via three electrical connections 254-258a-c respectively. The connection 248 and the connection 250 are generally identical. Similarly, each of the individual contacts 252a-258c is generally identical to each other and to the contact 248 and the contact 250. Each of the individual connections 248, 250, and 252-258a-c may be referred to as a standard connection.

To calibrate a calibrated test sensor 200′, as shown in FIG. 4b, ratios of conductivity are utilized. For example, the ratio of conductivity between the first contact 224 and the third contact 228 is compared to the conductivity between the second contact 226 and the fourth contact 230. The connection 248 of the test sensor 200 has been terminated in the calibrated test sensor 200′. Therefore, no conduction occurs between the first contact 224 and the third contact 228. The connection 250 remains between the second contact 226 and the fourth contact 230. At least one of the connection 248 and the connection 250 must remain intact. Thus, the conductivity between the second contact 226 and the fourth contact 230 is greater than the conductivity between the first contact 224 and the third contact 228. Therefore, the conductivities between the second contact 226 and the fourth contact 230 will be used to divide the conductivities between the other contacts 228-238 on the calibrated sensor 200′ and generate the ratioed conductivities.

Referring to the calibrated test sensor 200′, the conductivity between the fourth contact 230 and the sixth contact 234, the sixth contact 234 and the eighth contact 238 will be equivalent to the conductivity of the standard connection, as two of the three connections, the connections 254a, 254b, 258a, and 258b have been terminated, leaving only the connections 254c and 258c. Thus, the ratioed conductivity between the fourth contact 230 and the sixth contact 234 and the sixth contact 234 and the eighth contact 238 will be approximately one. As shown in the calibrated test sensor 200′, the ratioed conductivity between the third contact 228 and the fifth contact 232 is generally two, as the connection 252b and the connection 252c remain between the third contact 228 and the fifth contact 232. The conductivity between the fifth contact 232 and the seventh contact 236 is generally three times the standard connection, as all three connections 256a-256c remain. Therefore, the ratioed conductivity between the fifth contact 232 and the seventh contact 236 is generally three.

Thus, three possible ratioed conductivity values are present in the sensor 200 for every pair of contacts 228-238: the connection between the contacts may be generally identical to the standard connection, making the ratioed conductivity value generally one; the connection may be generally identical to twice the standard connection, making the ratioed conductivity generally two; or the connection between the pair of contacts may be generally identical to three times the standard connection, making the ratioed conductivity value three.

A similar calibration may be performed maintaining only the standard connection 248 between the first contact 224 and third the contact 228, and terminating the connection 250 between the second contact 226 and the fourth contact 230. It is further possible that both the standard connections 248, 250 will be maintained. Therefore, a total of 243 calibrations exist that can be used to calibrate the calibrated sensor 200′.

It is contemplated that a laser may be used to terminate the connections between the contacts in the above embodiments. It is also contemplated that the connections may be terminated by other processes.

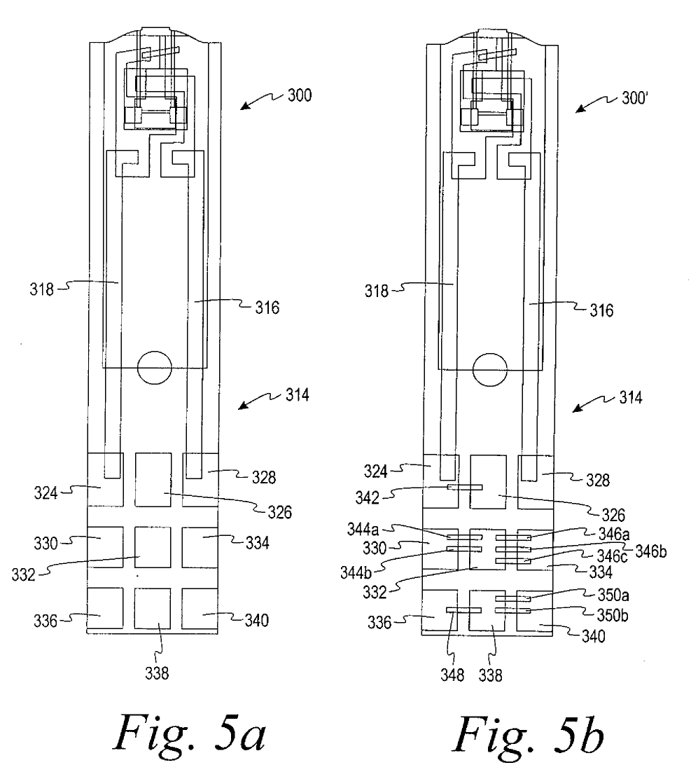

While the above embodiments have been described where connections are terminated, it is further contemplated that connections between contacts may be added to calibrate a test sensor. A test sensor 300 is shown in FIG. 5a that has a connection 316 to a working electrode, a connection 318 to a reference or counter electrode, and a meter contact area 314. The meter-contact area 314 comprises nine electrical contacts 324-340. The counter electrode is electrically connected to a first contact 324, while the working electrode 316 is electrically connected to a second contact 328. No additional electrical connection is present within the meter-contact area 314 of the test sensor 300. The first contact 324 and the second contact 328 are considered testing contacts, as they are used in determining the analyte level of the sample. The third contact 326, the fourth contact 330, the fifth contact 332, the sixth contact 334, the seventh contact 336, the eighth contact 338, and the ninth contact 340 are considered coding contacts, as they are used in coding a calibration on the test sensor 300.

Once the reactivity is determined, the sensor 300 may be encoded by printing electrical connections between the electrical contacts 324-340 utilizing conductive ink to create a calibrated test sensor 300′ as shown in FIG. 5b. An electrical connection 342 exists between the first contact 324 and the third contact 326. Thus, the connection 342 forms a standard connection that may be utilized when calibrating the calibrated test sensor 300′. As no connection is present between the second contact 326 and the third contact 328, the conductivity of connections between all the other contacts 330-340 will be divided by the conductivity of the connection 342 between the first contact 324 and the third contact 326 to give a ratioed conductivity.

Referring to the calibrated test sensor 300′, the conductivity between the seventh contact 336 and the eighth contact 338 will be equivalent to the conductivity of the standard connection, as a single connection 348 is present between the seventh contact 336 and the eighth contact 338. Thus, the ratioed conductivity will be approximately one. As shown in the calibrated test sensor 300′, the conductivity between the fourth contact 330 and the fifth contact 332 is generally twice the standard connection, as connections 344a and 334b have been added between the fourth contact 330 and the fifth contact 332. Similarly, the conductivity between the eight contact 338 and the ninth contact 340 of the calibrated test sensor 300′ is generally identical to twice the standard connection 342. Hence, the ratioed conductivity of both of these connections is about two. The ratioed conductivity between the fifth contact 332 and the sixth contact 336 is generally three, as three connections 346a-346c have been added

While the above described test sensors have been described with respect to electrochemical testing, it should be understood that the present invention is operable with optical testing systems or other testing systems.

The above described test sensors utilize a pre-printed blank or an uncoded label that is later encoded with calibration information. Calibration information is not known until after a sensor is printed, thus calibration information may not be coded onto a sensor until after the sensor is printed. Thus, according to some embodiments, encoding of calibration information is performed utilizing high-speed, low-cost processes, such as lasers or ink-jet printers located in fixed positions that mark blank sensors as the sensors pass by.

### ALTERNATIVE EMBODIMENT A

An electrochemical test sensor for determining the concentration of an analyte in a fluid sample, the electrochemical test sensor comprising:
a base;
a reagent layer;
a lid;
and a meter contact area having a plurality of contacts, a high conductivity electrical connection and a low conductivity electrical connection being formed between adjacent ones of the plurality of contacts, wherein at least one of the electrical connections in the meter contact area is terminated to encode calibration information on the test sensor.

### ALTERNATIVE EMBODIMENT B

The test sensor of Alternative Embodiment A, wherein the plurality of contacts has at least a first testing contact, a second testing contact, and at least four coding contacts.

### ALTERNATIVE EMBODIMENT C

The test sensor of Alternative Embodiment B, wherein at least one of the electrical connections between one of the testing contacts and one of the coding contacts is terminated.

### ALTERNATIVE EMBODIMENT D

The test senor of Alternative Embodiment C, wherein the calibration information comprises ratios of conductivity between adjacent ones of the coding contacts compared to a conductivity between one of the testing contacts and one of the coding contacts.

### ALTERNATIVE EMBODIMENT E

The test sensor of Alternative Embodiment B, wherein the first testing contact and two of the coding contacts are arranged in a first column, and the second testing contact and two of the coding contacts are arranged in a second column, and electrical connections are only present between contacts within the same column.

### ALTERNATIVE EMBODIMENT F

The test sensor of Alternative Embodiment A, wherein the reagent is adapted to react with glucose.

### ALTERNATIVE EMBODIMENT G

The test sensor of Alternative Embodiment F, wherein the reagent is glucose dehydrogenase.

### ALTERNATIVE EMBODIMENT H

The test sensor of Alternative Embodiment A, wherein the high conductivity electrical connections include silver.

### ALTERNATIVE EMBODIMENT I

The test sensor of Alternative Embodiment H, wherein the low conductivity electrical connections include carbon.

### ALTERNATIVE EMBODIMENT J

An electrochemical test sensor for determining the concentration of an analyte in a fluid sample, the electrochemical test sensor comprising:
a base;
a reagent layer;
a lid;
and a meter contact area having a plurality of contacts,
   the contacts having a first testing contact, a second testing contact and at least four coding contacts, at least a first electrical connection being formed between the first testing contact and a first one of the plurality of coding contacts, at least a second electrical connection being formed between the second testing contact and a second one of the plurality of coding contacts, and a plurality of electrical connections being formed between the plurality of adjacent coding contacts, wherein at least one of the connections in the meter contact area is terminated to encode calibration information on the test sensor.

### ALTERNATIVE EMBODIMENT K

The test sensor of Alternative Embodiment J, wherein at least one of the electrical connections between one of the testing contacts and one of the coding contacts is terminated.

### ALTERNATIVE EMBODIMENT L

The test senor of Alternative Embodiment K, wherein the calibration information comprises ratios of conductivity between adjacent ones of the coding contacts compared to a conductivity between one of the testing contacts and one of the coding contacts.

### ALTERNATIVE EMBODIMENT M

The test sensor of Alternative Embodiment J, wherein the first testing contact and two of the coding contacts are arranged in a first column, and the second testing contact and two of the coding contacts are arranged in a second column, and electrical connections are only present between contacts within the same column.

### ALTERNATIVE EMBODIMENT N

An electrochemical test sensor for determining the concentration of an analyte in a fluid sample, the electrochemical test sensor comprising:
a base;
a reagent layer;
a lid;
and a meter contact area having a plurality of contacts,
   the contacts having a first testing contact, a second testing contact and at least four coding contacts, at least a first electrical connection being added between the first reference contact and a first one of the plurality of coding contacts, and at least a second electrical connection being added between two of the plurality of adjacent coding contacts to encode calibration information of the test sensor.

### ALTERNATIVE EMBODIMENT O

The test senor of Alternative Embodiment N, wherein the calibration information comprises ratios of conductivity between adjacent ones of the coding contacts compared to a conductivity between one of the testing contacts and one of the coding contacts.

### ALTERNATIVE PROCESS P

A method of encoding calibration information onto a single test sensor adapted for use in determining a concentration of at least one analyte in a body fluid, the method comprising the acts of:
providing a test sensor having a plurality of electrical contacts, each of the plurality of electrical contacts being electrically connected to at least one adjacent contact via at least one connection;
determining a reactivity level of the test sensor;
terminating at least one connection electrically connecting two adjacent contacts; and
calculating a ratio of conductivities between pairs of adjacent contacts of the plurality of electrical contacts after the act of terminating, the ratios of conductivity providing information to a test meter regarding the calibration of the test sensor.

### ALTERNATIVE PROCESS Q

The method of Alternative Process P, wherein the plurality of electrical contacts has at least two testing contacts and at least four coding contacts and the act of terminating terminates a connection between a testing contact and a coding contact.

### ALTERNATIVE PROCESS R

The method of Alternative Process Q, wherein the act of terminating further terminates at least one connection between two coding contacts.

### ALTERNATIVE PROCESS S

The method of Alternative Process P, wherein the act of terminating utilizes a laser.

### ALTERNATIVE PROCESS T

A method of encoding calibration information onto a single test sensor adapted for use in determining a concentration of at least one analyte in a body fluid, the method comprising the acts of:
providing a test sensor having a plurality of electrical contacts;
determining a reactivity level of the test sensor;
forming at least one connection electrically connecting two adjacent contacts;
calculating a ratio of conductivities between pairs of adjacent contacts of the plurality of electrical contacts after the act of forming, the ratios of conductivity providing information to a test meter regarding the calibration of the test sensor.

### ALTERNATIVE PROCESS U

The method of Alternative Process T, wherein the plurality of electrical contacts has at least two testing contacts and at least four coding contacts and the act of forming creates a connection between a testing contact and a coding contact.

### ALTERNATIVE PROCESS V

The method of Alternative Process U, wherein the act of forming further creates at least one connection between two coding contacts.

### ALTERNATIVE PROCESS W

The method of Alternative Process T, wherein act of forming utilizes a printer for printing a conductive ink.

### ALTERNATIVE EMBODIMENT X

An electrochemical test sensor for determining the concentration of an analyte in a fluid sample, the electrochemical test sensor comprising:
a base;
a reagent layer;
a lid;
and a meter contact area having a plurality of contacts, a reference electrical connection being formed between adjacent ones of the plurality of contacts, wherein at least one additional electrical connection in the meter contact area encodes calibration information on the test sensor.

While the invention is susceptible to various modifications and alternative forms, specific embodiments and methods thereof have been shown by way of example in the drawings and are described in detail herein. It should be understood, however, that it is not intended to limit the invention to the particular forms or methods disclosed, but, to the contrary, the intention is to cover all modifications, equivalents and alternatives falling within the spirit and scope of the invention as defined by the appended claims.

1. An electrochemical test sensor for determining the concentration of an analyte in a fluid sample, the electrochemical test sensor comprising:
a base;
a reagent layer;
a lid;
and a meter contact area having a plurality of contacts, a high conductivity electrical connection and a low conductivity electrical connection being formed between adjacent ones of the plurality of contacts, wherein at least one of the electrical connections in the meter contact area is terminated to encode calibration information on the test sensor.

2. The test sensor of claim 1, wherein the plurality of contacts has at least a first testing contact, a second testing contact, and at least four coding contacts.

3. The test sensor of claim 2, wherein at least one of the electrical connections between one of the testing contacts and one of the coding contacts is terminated.

4. The test senor of claim 3, wherein the calibration information comprises ratios of conductivity between adjacent ones of the coding contacts compared to a conductivity between one of the testing contacts and one of the coding contacts.

5. The test sensor of claim 2, wherein the first testing contact and two of the coding contacts are arranged in a first column, and the second testing contact and two of the coding contacts are arranged in a second column, and electrical connections are only present between contacts within the same column.

6. The test sensor of claim 1, wherein the reagent is adapted to react with glucose.

7. The test sensor of claim 6, wherein the reagent is glucose dehydrogenase.

8. The test sensor of claim 1, wherein the high conductivity electrical connections include silver.

9. The test sensor of claim 8, wherein the low conductivity electrical connections include carbon.

10. An electrochemical test sensor for determining the concentration of an analyte in a fluid sample, the electrochemical test sensor comprising:
a base;
a reagent layer;
a lid;
and a meter contact area having a plurality of contacts,
the contacts having a first testing contact, a second testing contact and at least four coding contacts, at least a first electrical connection being formed between the first testing contact and a first one of the plurality of coding contacts, at least a second electrical connection being formed between the second testing contact and a second one of the plurality of coding contacts, and a plurality of electrical connections being formed between the plurality of adjacent coding contacts, wherein at least one of the connections in the meter contact area is terminated to encode calibration information on the test sensor.

11. The test sensor of claim 10, wherein at least one of the electrical connections between one of the testing contacts and one of the coding contacts is terminated.

12. The test senor of claim 11, wherein the calibration information comprises ratios of conductivity between adjacent ones of the coding contacts compared to a conductivity between one of the testing contacts and one of the coding contacts.

13. (canceled)

14. An electrochemical test sensor for determining the concentration of an analyte in a fluid sample, the electrochemical test sensor comprising:
a base;
a reagent layer;
a lid;
and a meter contact area having a plurality of contacts,
the contacts having a first testing contact, a second testing contact and at least four coding contacts, at least a first electrical connection being added between the first reference contact and a first one of the plurality of coding contacts, and at least a second electrical connection being added between two of the plurality of adjacent coding contacts to encode calibration information of the test sensor.

15. (canceled)

16. A method of encoding calibration information onto a single test sensor adapted for use in determining a concentration of at least one analyte in a body fluid, the method comprising the acts of:
providing a test sensor having a plurality of electrical contacts, each of the plurality of electrical contacts being electrically connected to at least one adjacent contact via at least one connection;
determining a reactivity level of the test sensor;
terminating at least one connection electrically connecting two adjacent contacts; and
calculating a ratio of conductivities between pairs of adjacent contacts of the plurality of electrical contacts after the act of terminating, the ratios of conductivity providing information to a test meter regarding the calibration of the test sensor.

17. The method of claim 16, wherein the plurality of electrical contacts has at least two testing contacts and at least four coding contacts and the act of terminating terminates a connection between a testing contact and a coding contact.

18. The method of claim 17, wherein the act of terminating further terminates at least one connection between two coding contacts.

19. (canceled)

20. A method of encoding calibration information onto a single test sensor adapted for use in determining a concentration of at least one analyte in a body fluid, the method comprising the acts of:
providing a test sensor having a plurality of electrical contacts;
determining a reactivity level of the test sensor;
forming at least one connection electrically connecting two adjacent contacts;
calculating a ratio of conductivities between pairs of adjacent contacts of the plurality of electrical contacts after the act of forming, the ratios of conductivity providing information to a test meter regarding the calibration of the test sensor.

21. The method of claim 20, wherein the plurality of electrical contacts has at least two testing contacts and at least four coding contacts and the act of forming creates a connection between a testing contact and a coding contact.

22. The method of claim 21, wherein the act of forming further creates at least one connection between two coding contacts.

23. (canceled)

24. An electrochemical test sensor for determining the concentration of an analyte in a fluid sample, the electrochemical test sensor comprising:
a base;
a reagent layer;
a lid;
and a meter contact area having a plurality of contacts, a reference electrical connection being formed between adjacent ones of the plurality of contacts, wherein at least one additional electrical connection in the meter contact area encodes calibration information on the test sensor.
